(19) United States
(12) Patent Application Publication
(10) Pub. No.: US 2010/0240915 A1
(43) Pub. Date: Sep. 23, 2010

(54) Pre-Treatment of Crude Alcohol or Furan Feed to a Vapor Permeation Apparatus

(76) Inventors: Robert Jansen, Portela (PT); Anthony Baiada, Dagenham (GB); John Kerr, South Croydon (GB); Peter Lloyd-Jones, Loudon, TN (US)

Correspondence Address: WILLIAMS, MORGAN & AMERSON, P.C., 10333 RICHMOND, SUITE 1100, HOUSTON, TX, 77042, US

(21) Appl. No.: 12/696,083

(22) Filed: Jan. 29, 2010

Related U.S. Application Data
(60) Provisional application No. 61/149,117, filed on Feb. 2, 2009.

(51) Int. Cl. C07D 307/36; C07C 29/76

(52) U.S. Cl. 549/506; 549/505; 568/700; 568/913

(57) ABSTRACT

We disclose a method of extracting an alcohol or furan from a predominantly liquid stream comprising the alcohol or furan, comprising removing cations from the predominantly liquid stream comprising the alcohol or furan, using a cation-exchange resin; removing anions from the predominantly liquid stream comprising the alcohol or furan, using an anion-exchange resin; and recovering alcohol or furan from the predominantly liquid stream comprising the alcohol or furan, using either a vapor permeation membrane, a perevaporation process, or both.

This application claims priority from U.S. provisional patent application Ser. No. 61/149,117, filed on Feb. 2, 2009, which is incorporated herein by reference.

### BACKGROUND OF THE INVENTION

The present invention relates generally to the field of recovery of alcohols or furans from a predominantly liquid stream. More particularly, it concerns use of cation- and anion-exchange resins prior to recovery of the alcohols or furans by use of a vapor permeation membrane.

### SUMMARY OF THE INVENTION

In one embodiment, the present invention relates to a method of extracting an alcohol or furan from a predominantly liquid stream comprising the alcohol or furan, comprising removing cations from the predominantly liquid stream comprising the alcohol or furan, using a cation-exchange resin; removing anions from the predominantly liquid stream comprising the alcohol or furan, using an anion-exchange resin; and recovering alcohol or furan from the predominantly liquid stream comprising the alcohol or furan, using either a vapor permeation membrane, a perevaporation process, or both.

In one embodiment, recovering uses a vapor permeation membrane.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a flowchart of one method according to the present invention.

FIG. 2 shows the concentration of various ions in an aqueous solution containing ethanol after ion exchange as described in Example 1.

### DESCRIPTION OF ILLUSTRATIVE EMBODIMENTS

In one embodiment, the present invention relates to a method of extracting an alcohol or furan from a predominantly liquid stream comprising the alcohol or furan, comprising: removing cations from the predominantly liquid stream comprising the alcohol or furan, using a cation-exchange resin; removing anions from the predominantly liquid stream comprising the alcohol or furan, using an anion-exchange resin; and recovering the alcohol or furan from the predominantly liquid stream comprising the alcohol or furan, using either a vapor permeation membrane, a perevaporation process, or both.

The alcohol or furan can be produced by any appropriate technique. One such technique is fermentation of a feedstock by an appropriate microorganism. Another such technique is a biomass-to-liquid technique, such as the Fischer-Tropsch process, flash pyrolysis, or catalytic depolymerization. Exemplary alcohol production techniques are given by The Alcohol Textbook K. Jacques, T. P. Lyons, and D. R. Kelsall, ISBN 1-897676-735. Exemplary furan production techniques are given by Kirk-Othmer Encycolpedia of Chemical Technology, Volume 6, p. 1005.

In one embodiment, the alcohol or furan is selected from the group consisting of ethanol, butanol, and 2,5-dimethylfuran.

Regardless of how it is produced, typically the alcohol or furan will be a component of a predominantly liquid stream. By “predominantly liquid” is meant that the stream comprises a liquid phase and a vapor phase in equilibrium with the liquid phase. The alcohol or furan may be the predominant component of the predominantly liquid stream or it may be in solution with a solvent. The solvent may be water or an organic solvent in which the alcohol or furan is soluble. The predominantly liquid stream may also contain other materials, such as traces of catalysts, traces of fermentation media, products of side reactions, and ions present in non-deionized water, among other materials. As a result, it may be desirable to purify the alcohol or furan from the other components of the predominantly liquid stream.

Although distillation techniques, such as vapor permeation or perevaporation, can be readily used to purify the alcohol or furan, a vapor prepared by heating the predominantly liquid stream may comprise, in addition to the alcohol or furan, various of the other materials referred to above. Such materials may deposit on vapor permeation membranes or perevaporation apparatus and impair the efficiency of recovery of the alcohol or furan. Therefore, removal of such materials prior to recovery of the alcohol or furan is desirable.

As stated above, the method comprises removing cations from the predominantly liquid stream comprising the alcohol or furan, using a cation-exchange resin. “Removing” and other verb forms thereof, as used herein, indicate that at least some of the cations (or anions) present in the predominantly liquid stream prior to performing a removing step are absent from the predominantly liquid stream after performing the removing step. In one embodiment, removing removes at least 50 mol % of the cations (or anions), such as at least 60 mol %, 70 mol %, 80 mol %, 90 mol %, 95 mol %, 99 mol %, 99.5 mol %, or 99.9 mol %. Use of resins to remove ions from a predominantly liquid stream can be performed by the person of ordinary skill in the art having benefit of the present disclosure as a matter of routine experimentation. Cation-exchange resins can be further defined as strong acid cation (SAC) resins or weak acid cation (WAC) resins. An SAC resin is a cation-exchange resin with a pKa less than 2. A WAC resin is a cation-exchange resin with a pKa of 2 to 7.

In one embodiment, the cation-exchange resin is an SAC resin.

Any cations present in the predominantly liquid stream can be removed during the removing step. In one embodiment, the cations are selected from the group consisting of iron, sodium, and mixtures thereof.

The countercation present in the resin prior to the removing step, which is exchanged for the cation during the removing step, can be any countercation whose presence in the predominantly liquid stream will have little if any tendency to deposit on vapor permeation membranes or the like. In one embodiment, the countercation is H.

The method also comprises removing anions from the predominantly liquid stream comprising the alcohol or furan, using an anion-exchange resin. Anion-exchange resins can be further defined as strong base anion (SBA) resins or weak base anion (WBA) resins. An SBA resin is an anion-exchange resin with a pKa of greater than 12. A WBA resin is an anion-exchange resin with a pKa of 7 to 12. In one embodiment, the anion-exchange resin is a weak base anion (WBA) resin.

Any anions present in the predominantly liquid stream can be removed during the removing step. In one embodiment, the anions contain sulfur. For example, the anions may be sulfate anions.

In one embodiment, the counteranion is Off.

Typically, the cation exchange step is performed before the anion exchange step.

Any resins known in the art can be used. The following summary indicates exemplary SAC, WAC, SBA, and WBA resins which are commercially available.

1) Strong acid cation—sulfonate (—SO₃H)

2) Weak acid cation—arboxlyate (—COOH)

3) Strong base anion—quaternary ammonium derivatives eg: Type 1 chloride form (—CH₂N(CH₃)⁺Cl⁻)

4) Weak base anion—tertiary amine-chloride form (—CH₂NHN(CH₃)₂⁺Cl⁻)

We have discovered that prior performance of the cation-exchange step and the anion-exchange step minimizes fouling of vapor permeation membranes. This shows clear economic benefit to the present method.

In addition to removing cations and anions by the use of appropriate resins, such materials can be further removed by other techniques. In one embodiment, the method further comprises filtering the predominantly liquid stream comprising the alcohol or furan after removing cations and removing anions and before recovering the alcohol or furan.

After cations and anions are removed from the predominantly liquid stream, the alcohol or furan are recovered from the predominantly liquid stream using either a vapor permeation membrane, a perevaporation process, or both. Both vapor permeation and perevaporation are known techniques and can be used by the person of ordinary skill in the art having the benefit of the present disclosure as a matter of routine experimentation.

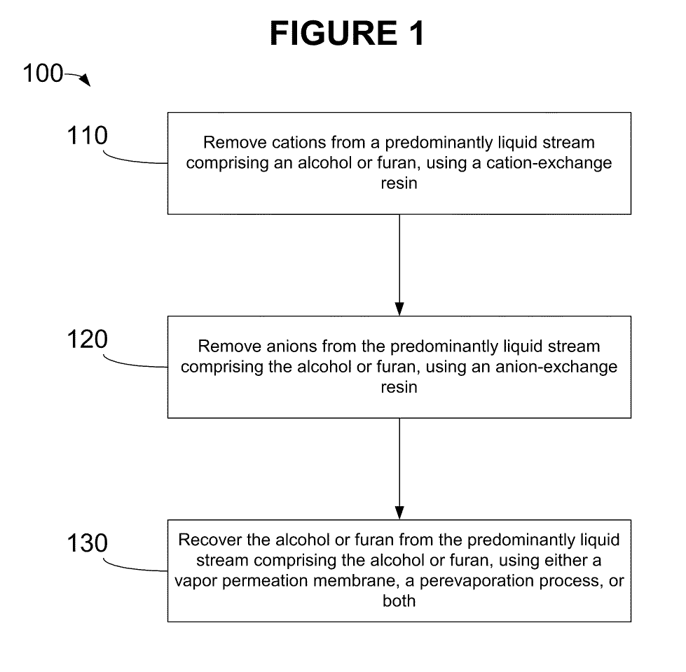

A flowchart of one embodiment of the method 100 is shown in FIG. 1. The method comprises a step of removing 110 cations from the predominantly liquid stream comprising the alcohol or furan, using a cation-exchange resin; removing 120 anions from the predominantly liquid stream comprising the alcohol or furan, using an anion-exchange resin; and recovering 130 the alcohol or furan from the predominantly liquid stream comprising the alcohol or furan, using either a vapor permeation membrane, a perevaporation process, or both.

As will be attested by the examples below, we discovered that typical foulant on vapor permeation membranes in ethanol production processes contains iron (Fe) and sulfur (S). We further discovered that use of both a cation-exchange resin and an anion-exchange resin allows use of vapor permeation membranes with only prefiltration to remove extraneous particulate matter in ethanol production processes. We also concluded that use of both a cation-exchange resin and an anion-exchange resin allows use of vapor permeation membranes with minimal prefiltration in production processes for other alcohols and for furans.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A sample of 43% ethanol in water was obtained from a commercial sample of a known ethanol production process. Cation and anion analysis (by ICP and Dionex, respectively) gave the data shown in Table 1 and Table 2:

TABLE 1
Cation analysis
Cations (ppm on sample)

| Ca | Cu | Fe | K | Mg | Mn | Na | P | S |
|---|---|---|---|---|---|---|---|---|
| 0.8 | 0.3 | 21.9 | 0.0 | 0.1 | 0.0 | 0.2 | 0.0 | 75.0 |

TABLE 2A
Anion analysis
Anions & acids (ppm on sample)

| Chloride | Sulphate | Sulphite | Phosphate | Oxalate | Citrate |
|---|---|---|---|---|---|
| 0 | 71 | 71 | 0 | 0 | 0 |

TABLE 2B
Anion analysis
Anions & acids (ppm on sample)

| Aconitate | Lactate | Acetate | Malate | Formate | Nitrite | Nitrate |
|---|---|---|---|---|---|---|
| 0 | 61 | 151 | 0 | 7 | 0 | 0 |

This ethanolic stream was fed to a cation resin column and an anion resin column set up in series. The first column contained 100 ml of C150S cation resin (macroporous poly(styrene sulphonate), Sulphonic acid functional groups, Purolite, Bala Cynwyd, PA), and the second column contained 150 ml of A500S resin (Macroporous polystyrene crosslinked with divinylbenzene, Type 1 Quaternary Ammonium functional groups, Purolite).

The ethanol stream was fed to these columns at the rate of 25 ml/minute (10 Bed Volumes per hour for the anionic resin).

Samples of the stream exiting the anionic resin column were analyzed at periodic intervals.

Results

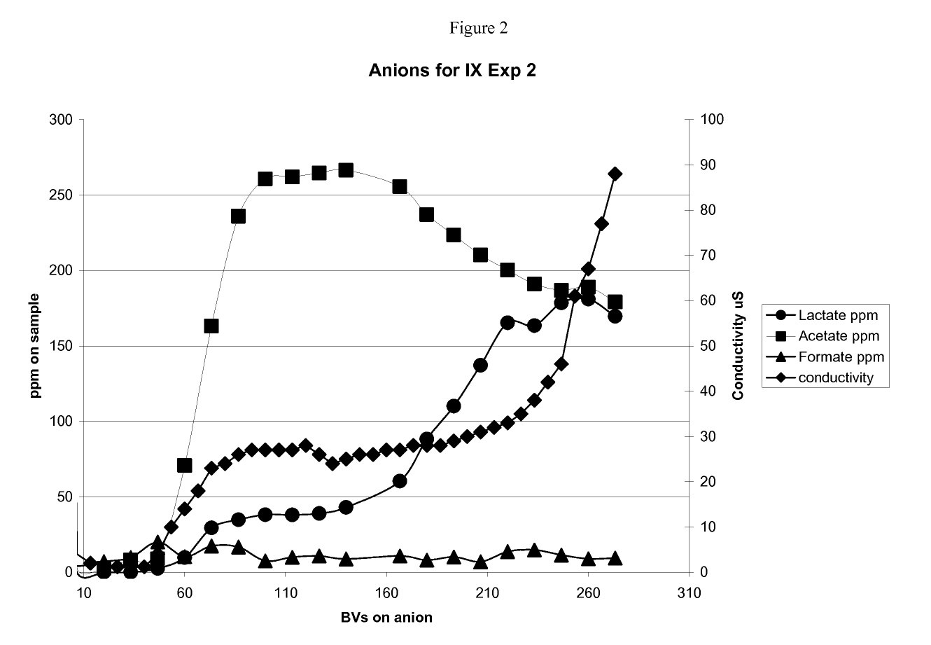

The anions measured in the product stream are reported in the FIG. 2, along with the conductivity (microSiemens/cm).

The feed stream contains a number of ions that can broadly be sub-divided into two categories: oxides of sulphur and carboxylic acids, the amounts of these ions can be expressed in terms of milliequivalents/litre of feed or as Equivalents/Bed Volume of feed:

| Species | Feed meq/litre | Eq/BV of feed |
|---|---|---|
| SO4 (2−) | 1.48 | 0.00022 |
| SO3 (2−) | 1.77 | 0.00027 |
| Total SO4 & | | 0.00049 |
| SO3 | | |
| Lactate (−) | 0.69 | 0.00010 |
| Acetate (−) | 2.56 | 0.00038 |
| Formate (−) | 0.16 | 0.00002 |
| Total | | 0.00100 |

FIG. 2 shows that the initial product from the ion exchange resins is very low in all anions. Though not to be bound by theory, we consider it likely that all anions are being retained on the active resin sites on the A500S resin (which has 1.15 Equivalents/litre). As the active sites on the resin sites become occupied, after about 50 BVs of feed, all the sites are full and the different anions compete for the available sites. As the carboxylic acid species are weaker acids than the sulphate and sulphite ions, they become displaced in the order of their pKas:

| Acid | pka1 |
|---|---|
| Formate (−) | 3.74 |
| Lactate (−) | 3.85 |
| Acetate (−) | 4.76 |

Whereas the stronger acids tend to be retained on the resin:

| Acid | pka1 | pka2 |
|---|---|---|
| SO4 (2−) | −3 | 1.99 |
| SO3 (2−) | 1.81 | 6.91 |

Thus, the sulfur species tend to be strongly absorbed on the anion resin and the composite product ethanol has a low sulfur content.

### Example 2

A sample of 37% ethanol in water was obtained. Cation and anion analysis (by ICP and Dionex respectively) gave the data shown in Table 3 and Table 4:

TABLE 3
Cation Analysis
Cations (ppm on sample)

| Ca | Cu | Fe | K | Mg | Mn | Na | P | S |
|---|---|---|---|---|---|---|---|---|
| 1.7 | 0.4 | 34.3 | 0 | 0 | 0 | 0.6 | 0 | 326 |

TABLE 4A
Anion Analysis
Anions & acids (ppm on sample)

| | | | Phos- | | | |
|---|---|---|---|---|---|---|
| Chloride | Sulphate | Sulphite | phate | Oxalate | Citrate | Aconitate |
| 1.4 | 28.1 | 111 | 0 | 0 | 0 | 0 |

TABLE 4B
Anion Analysis
Anions & acids (ppm on sample)

| Lactate | Acetate | Malate | Formate | Nitrite | Nitrate |
|---|---|---|---|---|---|
| 42 | 662 | 0 | 8 | 0 | 12 |

This ethanolic stream was fed to a cation resin column and an anion resin column set up in series. The first column contained 100 ml of C150S cation resin, whereas the second column contained 150 ml of A500S resin.

The ethanol stream was fed to these columns at the rate of 25 ml/minute (10 Bed Volumes per hour for the anionic resin).

Samples of the stream exiting the anionic resin column were analysed at periodic intervals.

### Example 3

We created an ultrapure ethanol stream to send to vapor permeation prefilters from an evaporator with a saturated tubesheet and to determine if any particulates were formed. We tested both 1 μm prefilters with ion exchange columns and a flooded evaporator, and 5 μm prefilters without ion exchange columns and with an unflooded evaporator.

The rectifier product is already a very clean stream (<5 μS) and this was diluted with fusels (yeast metabolic side products, typically including esters, ketones, and aldehydes) and demineralised water and then passed through ion exchange columns as described in Examples 1-2.

The evidence that the process is successful in removing trace impurities and preventing mechanical blockage of the VP prefilter were the prefilter dP trends in the plant. When IX resins were used before the vapor permeation membrane, the difference in pressure across the prefilter was minimal and stayed low. In the absence of IX resins, the pressure increased significantly across the prefilter over a period of time. Though not to be bound by theory, we submit the pressure increase was caused by particulate matter blocking (or blinding) the filter pores. Further evidence of this was provided by the fact that the prefilters were washed and virtually no solids were observed in the liquid washings, which contrasts to the non ion-exchanged ethanol where a layer of solids could be seen.

All of the methods disclosed and claimed herein can be made and executed without undue experimentation in light of the present disclosure. While the methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of extracting an alcohol or furan from a predominantly liquid stream comprising the alcohol or furan, comprising:
removing cations from the predominantly liquid stream comprising the alcohol or furan, using a cation-exchange resin;
removing anions from the predominantly liquid stream comprising the alcohol or furan, using an anion-exchange resin; and
recovering the alcohol or furan from the predominantly liquid stream comprising the alcohol or furan, using either a vapor permeation membrane, a perevaporation process, or both.

2. The method of claim 1, wherein the cation-exchange resin is a strong acid cation (SAC) resin or a weak acid cation (WAC) resin.

3. The method of claim 1, wherein the anion-exchange resin is a weak base anion (WBA) resin or a strong base anion (SBA) resin.

4. The method of claim 1, wherein the cations are selected from the group consisting of iron, sodium, and mixtures thereof.

5. The method of claim 1, wherein the anions contain sulfur.

6. The method of claim 1, wherein the alcohol or furan is selected from the group consisting of ethanol, butanol, and 2,5-dimethylfuran.

7. The method of claim 1, further comprising filtering the predominantly liquid stream comprising the alcohol or furan after removing cations and removing anions and before recovering the alcohol or furan.
